(19) United States
(12) Patent Application Publication
(10) Pub. No.: US 2008/0281115 A1
(43) Pub. Date: Nov. 13, 2008

(54) CHEMICAL MODIFICATION OF PARTIALLY HYDROGENATED VEGETABLE OIL TO IMPROVE ITS FUNCTIONAL PROPERTIES TO REPLACE PETROLEUM WAXES

(75) Inventors: Tong Wang, Ames, IA (US); Liping Wang, Solana Beach, CA (US)

Correspondence Address: MCKEE, VOORHEES & SEASE, P.L.C., 801 GRAND AVENUE, SUITE 3200, DES MOINES, IA, 50309-2721, US

(73) Assignee: IOWA STATE UNIVERSITY RESEARCH FOUNDATION, INC., Ames, IA (US)

(21) Appl. No.: 12/113,464

(22) Filed: May 1, 2008

Related U.S. Application Data
(60) Provisional application No. 60/917,510, filed on May 11, 2007.

(51) Int. Cl. C11B 3/02; C07C 53/00

(52) U.S. Cl. 554/149; 554/223; 554/167

(57) ABSTRACT

A novel, chemically-modified partially hydrogenated vegetable oil (PHVO) is described. The PHVO is produced by a three-step reaction process that includes epoxidation, a ring-opening reaction, followed by esterification. The modified PHVO has improved kneadability and, if mixed with fully hydrogenated fat(s), hardness comparable to unmodified PHVO.

### FIELD OF THE INVENTION

This invention relates to partially hydrogenated vegetable oil that is chemically modified with at least one functional group in its fatty acyl chain.

### BACKGROUND OF THE INVENTION

Petroleum paraffin wax is used in most commercial candles. It is the by-product from fractionation and refining of the fossil fuel crude oil. Although candles made from paraffin wax are typically inexpensive, consumers are becoming more interested in using “green” products or products from “green” processing. Fats of animal origin have long been used for making candles, but such candles are typically of lower quality because of the greasy texture, rancid odor, and sooting during burning. Partially hydrogenated vegetable oil (PHVO) may provide a promising replacement of petroleum wax and the traditional tallow wax because of its more desirable environmental and combustion properties. However, the inherent chemical structure and physical properties of PHVO limit its wide use in making a variety of types of candles. For example, the PHVO may be either too greasy or too brittle depending on the degree of hydrogenation, and lacks the cohesiveness that petroleum wax and beeswax possess.

The main defect of PHVO as candle material is its poor elasticity or cohesiveness, also referred to as kneadability compared to beeswax or paraffin wax. This may be attributed to the highly homogeneous molecular composition of the PHVO.

Accordingly, it is a primary objective of the present invention to provide a modified PHVO having better kneadability than conventional PHVO.

It is a further objective of the present invention to provide a modified PHVO that has hardability comparable to conventional PHVO.

It is a further objective of the present invention to provide a PHVO that is modified on the triacylglycerol acyl chain.

It is another objective of the present invention to provide a PHVO that is modified through the introduction of a hydroxyl group.

It is yet a further objective of the present invention to provide a PHVO that is chemically modified then combined with fully hydrogenated fats to further improve its hardness.

The method and means of accomplishing each of the above objectives as well as others will become apparent from the detailed description of the invention which follows hereafter.

### SUMMARY OF THE INVENTION

The present invention describes a chemically modified partially hydrogenated vegetable oil (PHVO) having improved cohesiveness and thermal properties. The PHVO is modified through the addition of functional groups into the fatty acyl chain. In this respect, the PHVO first undergoes an epoxidation reaction, followed by a ring-opening reaction. The resulting compound next undergoes esterification to provide a modified PHVO with improved properties.

The introduction of hydroxyl (OH) group significantly improves the cohesiveness of PHVO and increases its melting range. While the hardness of the modified wax lessens, its hardness can be significantly improved by mixing the modified wax with one or more unmodified PHVO.

### DETAILED DESCRIPTION OF THE DRAWINGS

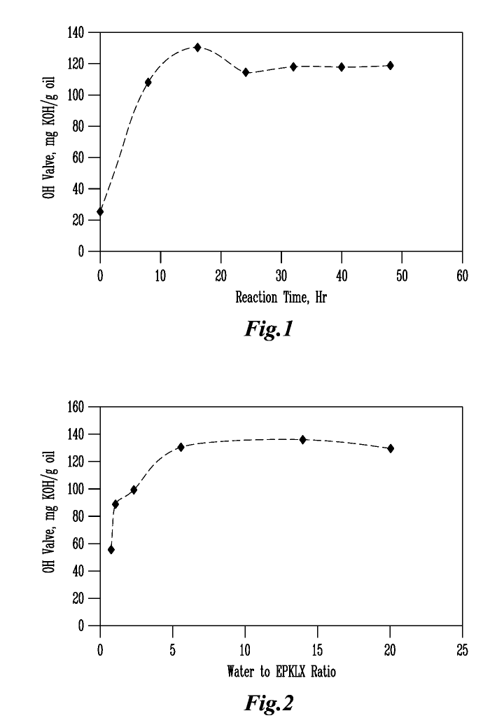

FIG. 1 illustrates the effect of reaction time on the oxirane ring opening reaction of epoxide, as described in Example 1.

FIG. 2 illustrates the effect of amount of water used on the oxirane ring opening reaction of epoxide, as described in Example 1.

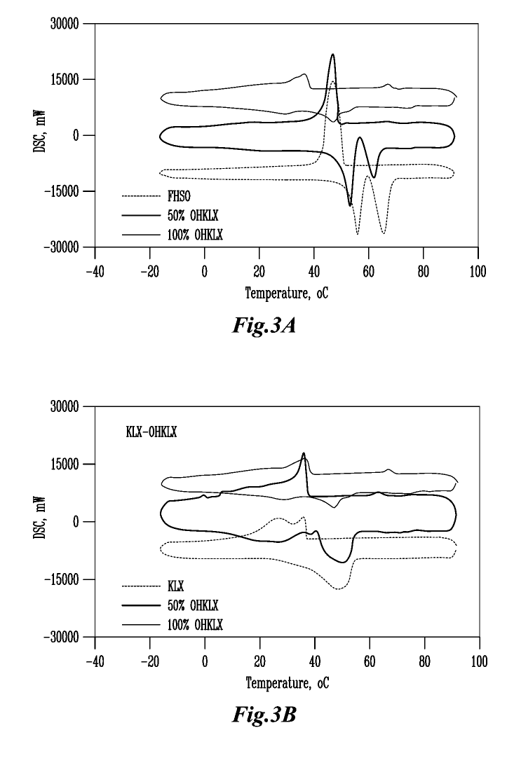

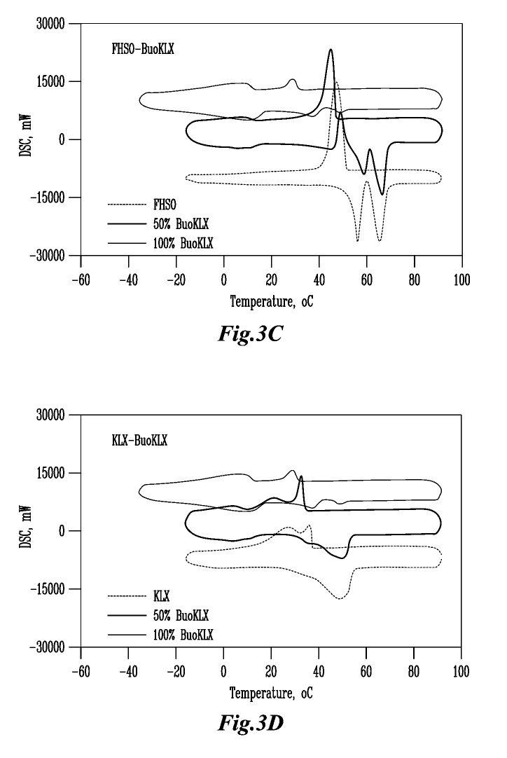

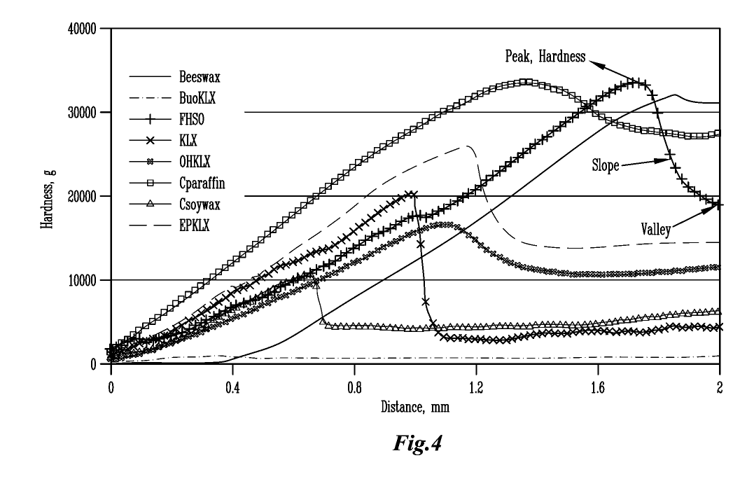

FIG. 3A-3D illustrate the melting and crystallization curves of wax mixtures FHSO-OHKLX (A), FHSO-BuoKLX (B), KLX-OHKLX (C), and KLX-BuoKLX (D).

FIG. 4 illustrates deformation profiles of waxes measured by a texture analyzer, as described in Example 1.

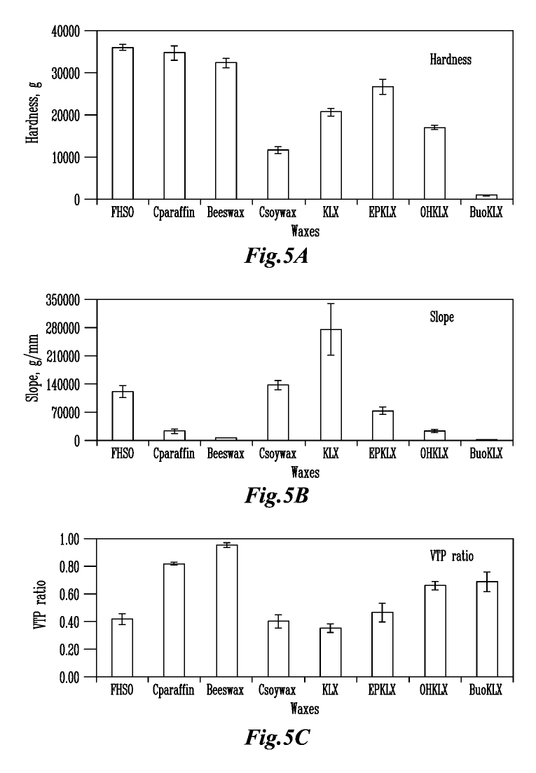

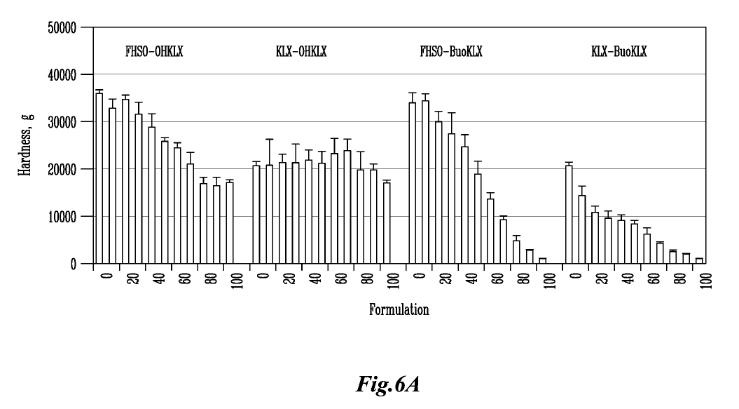

FIG. 5A-5C illustrate the textural properties of waxes measured by a texture analyzer, as described in Example 1.

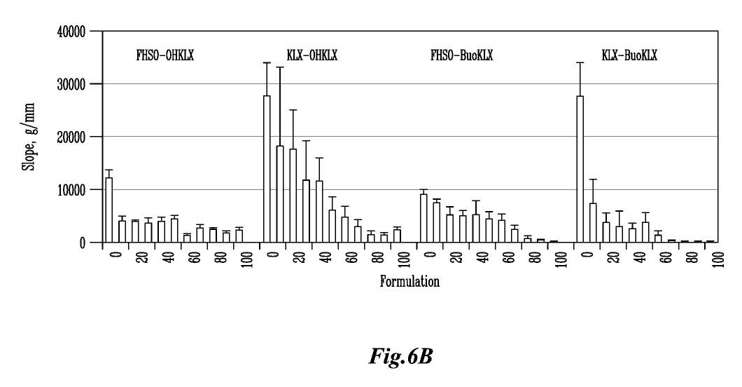

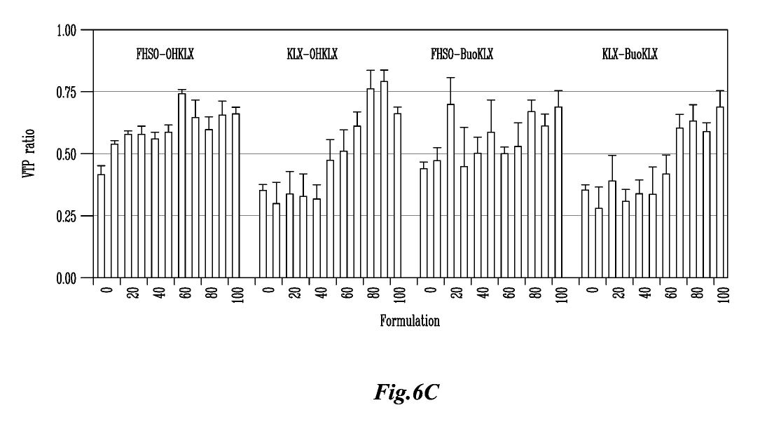

FIG. 6A-6C illustrate the hardness and cohesiveness/brittleness of wax mixtures, as described in Example 1. The number on X axis is the percentage of the modified wax in the mixture. Four series of mixtures presented.

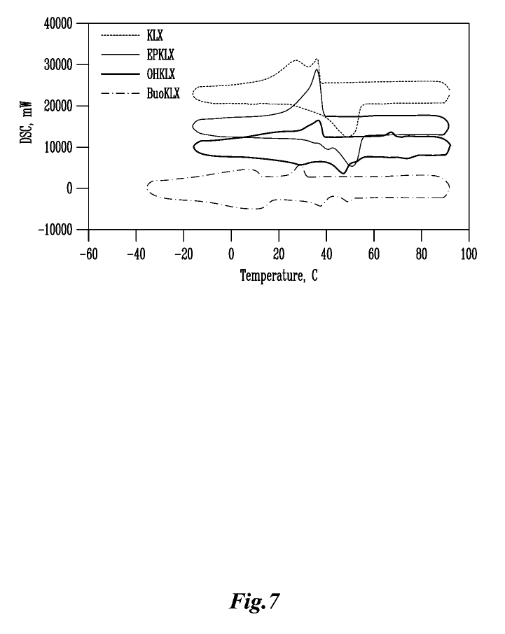

FIG. 7 illustrates the DSC profiles of KLX, EPKLX, OHKLX, and BuoKLX, as described in Example 1.

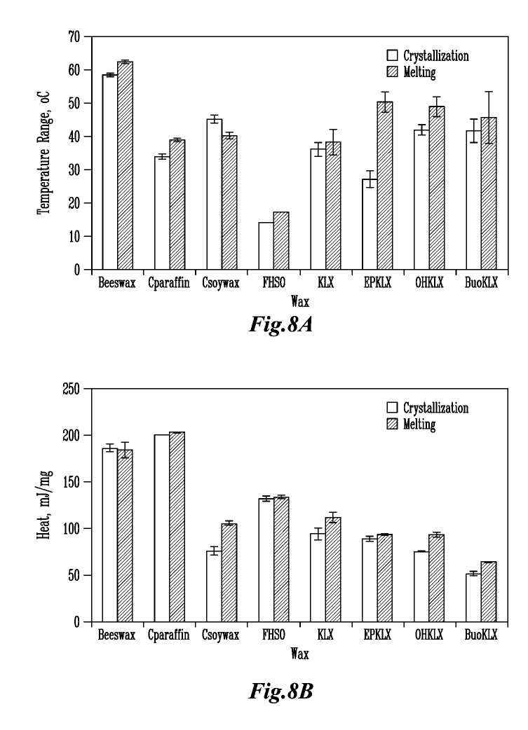

FIG. 8A-8B illustrate the temperature range and total heat change of waxes during crystallization and melting (A: Temperature range; B: Total heat of fusion), as described in Example 1.

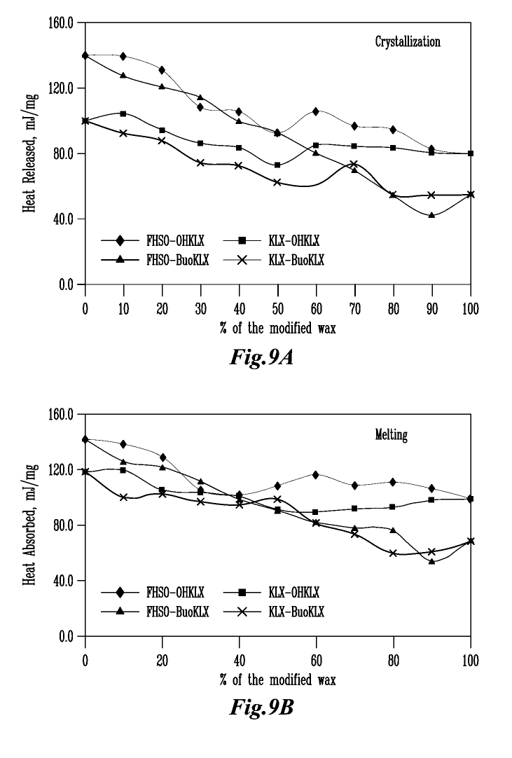

FIG. 9A-9B illustrate the melting and crystallization temperature ranges of wax mixtures as affected by the percentage of modified wax, as described in Example 1.

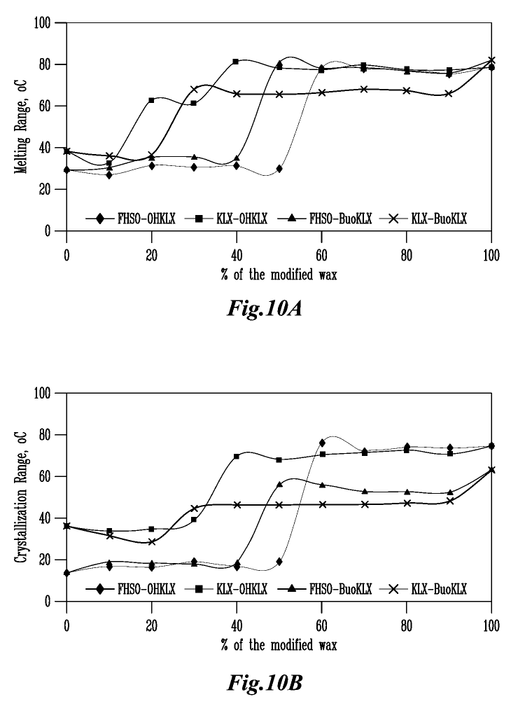

FIG. 10A-10B illustrate the total heats absorbed (B) or released (A) for the pair mixtures during crystallization and melting, as described in Example 1.

### DETAILED DESCRIPTION OF THE PREFERRED EMBODIMENT

The present invention relates to the development of a modified PHVO having improved cohesiveness and thermal properties through epoxidation, followed by a ring-opening reaction, then esterification whereby new functional groups are introduced into the triacylglycerol acyl chain. These newly synthesized derivatives may also be mixed with the fully hydrogenated base materials in order to improve the hardness of the modified PHVO.

Partially hydrogenated vegetable oils (PHVO), or trans fats, are essentially oils that have been chemically transformed from their normal liquid state (at room temperature) into solids. Trans fats are made when carbon (C) double bonded to another carbon in vegetable oil is reduced by “catalytic hydrogenation,” whereby a catalyst is used to add hydrogens to the structure of the fat molecule. Basically, hydrogen gas and a metal catalyst (often platinum or palladium) are reacted with a C═C to break their double bond, adding hydrogen (H) to each carbon. This process changes the oil from a cis formation into a trans formation. The resulting oil is a solid at room temperature and has a better shelf life than oils that are liquid at room temperature. The hydrogenation of vegetable oils creates a partially hydrogenated fat from the natural oils of soybeans, cottonseeds, peanuts, and rapeseeds, among others. It is expected that any PHVO or trans fat having a carbon-carbon double bond may be modified in accordance with the teachings of the invention.

The method of the invention first involves epoxidation of the PHVO by reacting the PHVO with an oxidant, with or without the presence of a solvent, and in the presence of a catalyst. The oxidant is generally an organic hydroperoxide such as t-butyl hydroperoxide or hydrogen peroxide; examples of oxidants include t-butyl hydroperoxide (max yield at pH 7.5-8.0), cumene hydroperoxide, hydrogen peroxide (max yield at pH 5.5), and urea-hydrogen peroxide complex. Hydrogen peroxide (H₂O₂) is a preferred oxidizing agent for use in the invention. The solvent may be an aqueous solvent composed of a buffer having a pH from about 4.5 to about 9.5 (preferably a pH from about 5 to about 9) such as aqueous phosphate buffer/Tween or water with a manual or automatic monitoring system that detects and adjusts the pH or hydrogen ion concentration; the solvent may be a nonpolar solvent such as benzene, hexane, acetonitrile, heptane, isooctane, dichloromethane, or toluene. The reaction time is about one minute up to about seven days (preferably about two hours to about 24 hours, more preferably about six hours to about 12 hours) depending upon the temperature, whereby the temperature coefficient (Q10) is 2. In this regard, at a temperature of about 55° C. the reaction time will be about 7 hours. In general, the reaction temperature will range from about 5° C. to about 75° C., preferably about 35° C. to about 75° C., with about 55° C. being most preferred.

Preferably, the oxidant is added to the PHVO in batches rather than all at once. A convenient method is to add (generally manually) oxidant every one or two hours; an automatic addition of small amounts of oxidant more frequently is possible. As the reaction progresses, the rate of conversion of starting material to product slows, and the oxidant may be added less frequently, or alternatively if automatic addition is used then the oxidant may be added more slowly. The amount of oxidant added is an amount sufficient to achieve maximum conversion to epoxide, although for some applications incomplete conversion to epoxide is desirable (in which case less oxidant is added).

As noted, the PHVO is combined with the oxidant in the presence of a catalyst. The catalyst is preferably a weak organic acid, such as acetic acid, formic acid, propionic acid, lactic acid, sorbic acid, and butyric acid. However, a stronger acid, such as sulfuric acid will also work as a catalyst for this purpose. An amount of catalyst is added that is sufficient to cause conversion of starting material to epoxide. Such quantities are readily ascertained by persons skilled in the art. At the end of the epoxidation reaction, the reaction medium is physically separated from the epoxide product. If the reaction medium is an organic solvent, the organic solvent and is removed by conventional means, such as evaporation, to give the epoxide product.

Following the epoxidation reaction, the PHVO undergoes a reaction to open the oxirane ring produced in an aqueous solution under acidic conditions. In this regard, the PHVO is combined with a strong acid, such as perchloric acid, sulfuric acid, sulfurous acid, or hydrochloric acid. Perchloric acid is preferred for purposes of this invention. The amount of acid used will vary depending upon the acid(s) selected from use, as well as other factors such as temperature, but should be a concentration sufficient to effect ring-opening of the epoxide. In this regard, a water to PHVO ratio ranging from about 0.8-20 is preferably employed, with a ratio of from about 5-10 being preferred. Although reaction temperatures of from 20° C. to 150° C. may be utilized, the temperature range generally preferred is from 60° C. to 120° C. The reaction time required will vary as a consequence of the several interrelated variables affecting the rate of reaction. For the most part, however, reaction times of from 0.25 to 48 hours will be sufficient, with about 20 hours being preferred.

The resulting compound may be esterified by reacting with a fatty acid entity capable of incorporating fatty acid acyl groups onto the modified PHVO compound, said entities being selected from the group consisting of fatty acids, fatty acid esters, fatty acid anhydrides and fatty acid halides, with short-chain (C2-C5) fatty acid anhydrides being preferred. The modified PHVO is first dissolved in an organic solvent, such as methylene chloride (CH₂Cl₂). The dissolved compound is then reacted with the fatty acid entity, preferably in the presence of from about 0.001-1.0 mol of one or more fat-soluble nitrogen-containing base catalysts such as, but not limited to, dimethylaminopyridine (DMAP), tri-ethylamine, 4-pyrrolidinopyridine (PPY) and/or 4-(N,N-diallylamino)pyridine polymers (DAAP).

The compounds are reacted for a time and at a temperature sufficient to accomplish substantially complete (i.e., greater than 67%, more preferably, greater than 90%) esterification of the hydroxyl groups on the fatty acid chain of the modified PHVO compound. The reaction temperature is preferably from about 100° C. to about 350° C., with reaction times of from about 0.5 to 48 hours being generally sufficient to accomplish substantially complete esterification of the hydroxyl groups. A co-product having the structure HOR′ (i.e., water or an alcohol) will be generated as the esterification proceeds. To drive the reaction to completion, it is desirable to remove the co-product from the reaction mixture as it forms by a suitable method such as distillation or vacuum stripping. To accomplish substantially complete esterification of the intermediate compound, at least about 1 (more preferably, at least about 1.1) equivalent of the fatty acid entity per equivalent of hydroxyl groups in the fatty acid chain is used. For reasons of economy, it is preferred to utilize not more than about 3 equivalents of fatty acid entity per equivalent of hydroxyl groups. One the desired degree of esterification is achieved, the reaction is preferably quenched with ammonium chloride (NH₄Cl) or other suitable compound which binds with the catalyst and stops the reaction.

Once the desired degree of esterification has been accomplished, any residual unreacted fatty acid is preferably removed from the esterified PHVO. Suitable methods include vacuum steam stripping (distillation) at an elevated temperature (as described, for example, in U.S. Pat. No. 4,983,329), alkali neutralization to precipitate fatty acid salts which may then be removed by filtration, extraction (with methanol, for example), and dilution with a solvent such as hexane in which the desired product is soluble and the fatty acid is insoluble followed by filtration.

The resulting modified PHVO may either be used by itself for candle-making or other appropriate uses. While the PHVO modified in accordance with the invention has improved cohesiveness, its hardness is lower than that of the original material and those of commercial paraffin wax and beeswax. The melting range of PHVO increases after the modifications. By mixing the hydroxyl PHVO with the fully hydrogenated fats, the hardness is significantly improved, while the cohesiveness decreases with the increase in the hard fat addition. The formulation can be optimized to create the appropriate level of cohesiveness and hardness depending upon the concentration of fully hydrogenated fat, such as KLX and/or FHSO, if any, with which it is combined.

The following examples are offered to illustrate but not limit the invention. Thus, they are presented with the understanding that various formulation modifications as well as method of delivery modifications may be made and still be within the spirit of the invention.

### Example 1

### Synthesis of Modified Partially Hydrogenated Vegetable Oil

### Experimental Procedures

Materials. KLX™ (a commercial PHVO with 28.3% palmitic, 24.5% of stearic, and 48.7% of oleic acid) was provided by Loaders Croklaan (Channahon, Ill.) and fully hydrogenated soybean oil (FHSO) (11.6% palmitic and 88.4% stearic) was from Uniqema (Chicago, Ill.). Beeswax was provided from Strahl & Pitsch Inc. (West Babylon, N.Y.). Commercial paraffin (Cparaffin) candle was purchased from local grocery store. Commercial soywax (Csoywax) was from Soy Basics (New Hampton, Iowa).

Hydrogen peroxide (H2O2, 30% aq.), glacial acetic acid, perchloric acid (70% aq.), acetic anhydride, potassium hydroxide, hydrogen bromide (40-48% aq.), ammonium chloride, and organic solvents were from Fisher Scientific (Pittsburgh, Pa.). Butyric anhydride, Amberlite IR120H, dimethylaminopyridine (DMAP), and triethylamine were all purchased from Sigma-Aldrich (St. Louis, Mo.).

Epoxidation of KLX. Method reported by Park et al. (2004) was used for the synthesis of epoxidized KLX (EPKLX) with or without the presence of solvent. The KLX (121 g, 0.14 mol), glacial acetic acid (7.8 g, 0.13 mol), catalyst Amberlite IR-120 H (25 g) were placed in a round bottom, three-necked flask equipped with a mechanical stirrer, thermometer, and reflux condenser. The mixture was heated to 55° C., then 30% aq. H₂O₂ (23.8 ml, 0.21 mol) was added dropwise from a separatory funnel and the reaction was allowed at 55° C. for 7 or 24 hr. After the reaction was complete, the crude product was filtered immediately to remove the catalyst. The liquid was washed with hot distilled water several times till pH was near 7.0. When the reaction was conducted with solvents, such as benzene, hexane, and acetonitrile in the amount of 40 mL, the solvent was removed with a vacuum evaporator. To calculate peroxide conversion rate, the oxarine oxygen value was measured using the standard method AOCS Cd9-57, and the calculated oxirane oxygen was based on the oleic acid content. Therefore,

Peroxide conversion, %=Measured oxirane oxygen*100/Calculated oxirane oxygen).

In this study, sulfuric acid was also tested as catalyst in the non-solvent reaction system because it is a cheap and readily available catalyst. The amount of sulfuric acid added was 0.1% of KLX.

Ring-opening reaction of EPKLX. Ring-opening reaction was conducted to produce dihydroxy EPKLX (OHKLX) using the method reported by Sharma et al. (2006) with modification. The reaction was carried out by refluxing EPKLX in perchloric acid (2% of EPKLX) aqueous solution at 95° C. Perchloric acid was added dropwise under constant stirring. Two reaction parameters, time and amount of water, were examined. Samples were taken at various reaction times (0-48 hr) to determine optimal time for the reaction when water to EPKLX ratio of 20 was used. The other factor examined was the water to EPKLX ratio (0.8-20) at 16 hr of reaction. The optimal conditions were used to synthesize large quantity of OHKLX for testing and further reaction.

The OH values of OHKLX were measured to monitor the reaction using AOCS official method Tx 1a-66.

Esterification of OHKLX with butyric anhydride. To synthesize butyric ester of OHKLX (BuoKLX), OHKLX (0.1 mol) was dissolved in dry CH₂Cl₂ (100 ml) and stirred at ambient temperature. Butyric anhydride (0.1 mol) was added followed by 4-dimethylaminopyridine (DMAP) (0.01 mol) and tri-ethylamine (0.4 mol). After 6 hr, the reactants were quenched with saturated aq. NH₄Cl and the mixture washed with water. The solvent was removed by the rotary evaporation.

Structural confirmation by NMR. Structure of synthesized waxes was characterized using a Varian VXR 400 NMR (Varian, Polo Alto, Calif.) with a Bruker magnet (Bruker BioSpin, Billerica, Mass.). ¹H NMR (400 MHz, CDC13): KLX, δ=5.3 ppm (3H, —CH═CH—), δ=5.23 ppm (1H, —CH2-CH—CH2 of glycerol backbone); EPKLX, δ=2.6-2.9 ppm (3H, epoxy group), δ=2.3 ppm (6H, O═C—CH—), δ=5.23 ppm (1H, —CH2-CH—CH2 of glycerol backbone); OHKLX, δ=3.5 ppm (2.6H, OH—HC—HC—OH); BuoKLX, δ=5.23 ppm (1H, —CH2-CH—CH2 of glycerol backbone), δ=5.0 ppm (2.7H, CH—OOCC3H7). ¹³C NMR (400 MHz, CDC13): KLX, δ=130 ppm (C═C); EPKLX, δ=58.5 ppm (epoxide group); OHKLX, δ=73-75 ppm (OH—C—C—OH); BuoKLX, δ=172 ppm (—OCOC3H7).

Observation of fat crystalline by Polarized Light Microscopy. The microstructure of waxes was observed with the Polarized Light Microscopy (PLM). In order to erase the crystal memory of waxes, samples were heated in oven at 85° C. for 30 min. The glass microscopy slides and capillary tubes were also preheated in oven at 85° C. Small amount of melted wax (about 50 μL) was placed on a preheated glass slide. A second preheated glass slide was slipped over to produce a film with uniform thickness. Bubble entrapment was avoided during the slide preparation. The prepared slides were stored at room temperature until use. The slides were analyzed with a Zeiss Axioskop upright microscope (Carl Zeiss, Thornwood, N.Y.). A 20× Neofluar dry objective with numerical aperture (NA=0.50) was chosen in imaging mode. A dry turret condenser (NA=0.9) was used for differential interference contrast (DIC) microscope. Images were acquired with a camera system and processed with Adobe Photoshop software.

Hardness and cohesiveness measurements. The most important physical or textural properties to be modified and measured in this research are hardness and elasticity or cohesiveness which is the ability of deformation under applied force (Marangoni et al., 2005). To measure the hardness and cohesiveness of waxes, a TA.XT2i Texture Analyzer™ (Stable Micro Systems, Godalming, England) was used with a TX plate probe to measure the force during compression. The parameters of the method were as follow: pre-test and post-test speed of 2.0 mm/s, probe movement speed of 0.5 mm/s, and compression distance of 2.0 mm. Bulk waxes and wax mixtures were individually melt at 80° C. and formed 13.8×5 mm sample disks using the bottom plate of tube rack as a mold. The disks were stored at 4° C. for 24 hr before use. The highest compression force was taken as the hardness measure. The rate of deformation (slope, after reaching the peak) and the force ratio between the lowest and the highest points (valley to peak ratio, VTP) during deformation were used as the cohesiveness or brittleness measure of wax. The lower the value of the slope and the higher the value of VTP ratio, the more cohesive the material is. Two batches of wax materials were prepared, and three wax discs were made from each material for triplicate sample measurements.

Melting and crystallization properties. A differential scanning calorimeter (DSC 6200, Seiko Instruments, Inc., Shizuoka, Japan) equipped with a cooling controller using liquid N2 was used to measure melting and recrystallization properties. A sample of 6.0-9.0 mg was used. The temperature program reported by Rezaei et al (2002b) was adopted in this experiment. Briefly, an initial 2 min hold at 30° C. was followed by 30° C./min heating to 90° C. and with a second 5 min hold at 90° C. Then the sample was cooled to −20° C. at 10° C./min rate with a 10 min hold at −20° C. Heating was performed at 10° C./min rate to 90° C. The data collected in the last two steps were used to analyze the melting and crystallization properties of waxes. Each sample was measured once, and all treatments (individual waxes or mixtures) were duplicated.

Effect of the newly synthesized fats on physical properties of the base material. OHKLX was mixed with individual KLX and FHSO in weight percentage from 0 to 100. Similarly, BuoKLX was also mixed with individual KLX and FHSO. Each mixing and formulation was performed in duplicate. All physical and thermal properties described above were evaluated for these samples.

Statistical Analysis. ANOVA of the Statistical Analysis System (SAS) 9.1 (SAS institute, Cary, N.C.) was used for data analysis (SAS procedure guide, 2006). Mean and least significant differences (LSD) were determined.

### Results and Discussion

Synthesis of EPKLX. In the commercial epoxidation process of soybean or other oils, hydrogen peroxide is reacted with formic acid without catalyst if hydrogen peroxide is concentrated (50-70%), or with acetic acid using a strong acid as catalyst (Swern, 1971). Two most common catalysts for peroxyacetic acid formation are sulfuric acid and cross-linked polystyrene sulfonic acid cation-exchange resins. Sulfuric acid is the cheapest catalyst used on commercial scale. However, it causes molecular cleavage and waste disposal problems (Swern, 1971). Acidified cation exchange resin can be removed by filtering after the reaction and can be recycled on a large scale reaction. Solvents are not ordinarily used on industry scale production. But in most reported epoxidation reactions, benzene and toluene are widely recommended as dilute agents, particularly when sulfuric catalyst is used (Formo, 1982; Park, 2004). Any amount of solvent residue may be a toxic issue during candle burning or when the material is used for other purposes, therefore, solvent is not desirable in this reaction.

In this study, three solvents and two catalysts were selected and compared, and oxarine value was determined to optimize the epoxidation reaction (Table 1). When Amberlite IR-120H was used as the catalyst, the highest peroxide conversion (98.4%) was obtained in a non-solvent system, whereas a 90.3% conversion was obtained using the method reported by Park et al (2004), in which benzene was used as solvent or dilute agent. The peroxide conversion was only 79.4% using sulfuric acid as catalyst in a non-solvent system. Hexane and acetonitrile were also used to replace benzene, however, the peroxide conversions were both lower than that of benzene after 7 hr reaction. This result may be due to the slower reaction rate because the peroxide conversion increased by 25% as the reaction time was extended to 24 hr using acetonitrile as solvent.

TABLE 1
Epoxidation reaction of KLX with different solvent and catalyst

| Catalyst | Solvent | Reaction time (h) | Conversion (%) |
|---|---|---|---|
| Amberlite IR-120H | Benzene | 7 | 90.3 |
| | Hexane | 7 | 49.2 |
| | Acetonitrile | 7 | 62.7 |
| | Acetonitrile | 24 | 87.0 |
| | No solvent | 7 | 98.4 |
| Sulfuric acid | No solvent | 7 | 79.4 |

Swern (1971) reviewed the industrial procedures of epoxidation of soybean oil and pointed out that solvent was generally not required in epoxidation reactions utilizing ion-exchange-resin catalysts. In this study, EPKLX was prepared in a large scale using Amberlite IR120H as the catalyst in a non-solvent system. The oxirane oxygen value of this product was 2.35±0.02%. The oxirane conversion was as high as 98.4±0.8%. ¹H NMR of EPKLX also showed that the epoxidation reaction was practically complete. The C═C (δ=5.3, 3H) disappeared and epoxide group (δ=2.6-2.9 ppm, 3H) was produced.

Synthesis of OHKLX. The oxirane ring is more reactive than C═C and can be hydrolyzed into two OH groups in the hot acidic solution. In the method reported by Sharma et al. (2006), epoxidized vegetable oil was reacted with large amount of water (20:1, water: EPKLX, wt/wt) at 100° C. for 48 hr using perchloric acid as catalyst. To improve reaction efficiency, the reaction time and amount of water were optimized in this study. The OH values of products were measured to monitor the reaction. FIGS. 1 and 2 show the effect of reaction time and water on ring-opening reaction of the epoxide. The OH value of OHKLX increased to the highest at 16 hr, and then decreased after prolonged time. The longer reaction time may have caused molecular cleavage under the hot acidic condition (Rangarajan et al., 1995). By comparing the ¹H NMR data of individual OHKLX samples, hydrogen number at δ=3.5 ppm (OH—HC—HC—OH) was lower for the 48 hr-product (2.3H) than for the 16 hr-product (2.6H). The OH value decreased considerably as the amount of water was reduced to below about 5 times of EPKLX. The difference in the concentration of perchloric acid may account for such result. As a strong oxidizing agent, perchloric acid at high concentration might enhance the degradation of the newly formed OH group.

In the method reported by Sharma et al. (2006), chloroform was used to extract the organic material. Such toxic solvent has to be completely removed after the extraction. It took considerable amount of time to remove the solvent from OHKLX and it foamed during the process. In the preliminary experiment, it was also found that OH value and viscosity of final product decreased as OHKLX was evaporated of solvent at high temperature (80° C.) for a long time. The possible reason is the degradation of OH group when heated. In this study, water wash and crystallization in cold water were applied to avoid any side reaction and for improved efficiency.

Synthesis of BuoKLX. Sharma et al. (2006) synthesized butyric ester of dihydroxy soybean oil in pyridine, which was used as both the solvent and the base catalyst. The odor from any residual pyridine would make this process highly unsuitable for a candle application. In this experiment, methylene chloride was used as solvent and DMAP as the catalyst. A soft yellowish product was produced. The reaction was complete as suggested by NMR data. Peak for OH—HC—HC—OH at δ=3.5 ppm disappeared whereas peak for —CH—OOCC3H7 at δ=5.0 ppm was observed after reaction.

Observation of fat crystallines by Polarized Light Microscopy. PLM images were captured to compare the crystallization of waxes. FHSO had the most ordered crystallization. Beeswax had a fine and somewhat random crystallization. KLX and EPKLX had more ordered crystallization than Csoywax. Csoywax seemingly had similar image as beeswax, but it is a much softer and lower-melting product. After EPKLX is hydrolyzed to give hydroxy derivatives, the OHKLX had much finer and more random crystallization than the EPKLX, and it had similar image as that of beeswax. Few studies have been reported on PLM images of fats and waxes. Edwards (1957) studied the crystal habit of paraffin wax. Dorset (1995, 1999) demonstrated that beeswax had disordered lamellar interface. Although PLM doesn't provide quantitative and definitive comparison among materials of different source and with wide melting range, it does give a visual microscopic observation that may relate to wax's physical properties.

Hardness and cohesiveness. The hardness and deformation properties of bulk waxes were shown in FIGS. 4 and 5. The peak value of deformation curve in gram was used to represent the hardness of waxes (FIG. 4). Because the wax samples were hard and brittle, their cohesiveness could not be determined with the cone penetrometry method as used in the measurement of the rheological properties of cheese or butter (Wright et al., 2001). In the plate compression method used, the ratio between the valley value (lowest force) and the peak value (VTP ratio) and the absolute value of slope (the deformation rate) in g/mm provide good comparison for cohesiveness and brittleness among the wax samples from preliminary evaluations (FIG. 4) because they correlated with direct hand touch evaluation. A wax having higher VTP ratio and lower slope value is more cohesive and less brittle than a wax with lower VTP ratio and higher slope.

Hardness was significantly different among 8 wax samples (FIG. 5A-5C). FHSO had the hardest texture whereas BuoKLX, having semi-solid texture, was the softest waxes. Cparaffin wax and beeswax were harder than Csoywax, KLX, and the synthesized waxes. EPKLX had intermediate hardness which was greater than those of OHKLX and KLX. OHKLX was significantly softer than Cparaffin wax, beeswax, and KLX, but it was harder than Csoywax. Brittleness and cohesiveness also varied significantly among waxes (FIG. 5). Cparaffin and beeswax had higher compression force for longer time than other waxes (low slope and high ratio values). Csoywax and KLX, however, were deformed with the least compression in shortest time (high slope and low ratio values). The synthesized wax OHKLX and BuoKLX broke down much slower (low slope and high ratio values) than other triacylglycerol type of materials. The slope and VTP ratio data shown in FIG. 5 indicate that OHKLX has much improved cohesive properties compared to the unmodified vegetable waxes. If a more saturated material was used for the derivatization reaction, the hydroxy product will be harder. In addition, if a longer chain acid was used to synthesize the diester, the hardness of the ester will be higher. Thus, these reactions provide much promise in producing modified fats with desired properties.

OHKLX and BuoKLX are proved to be possible components for candle-making since they had better cohesiveness than the unmodified KLX. However, they lack the proper hardness when used alone. To improve the hardness of the newly synthesized materials, FHSO and KLX were mixed with the two modified waxes individually. Hardness, slope, and VTP ratio were measured to evaluate the textures of mixtures (FIG. 6A-6C). The hardness of the OHKLX was significantly affected by the addition of FHSO, but it was not as significantly affected by the addition of KLX. The hardness of BuoKLX was significantly improved by the addition of both FHSO and KLX. The addition of FHSO into OHKLX or BuoKLX increased the hardness of mixtures more significantly than the addition of KLX to the two modified waxes.

The cohesiveness of the synthesized waxes was significantly decreased with the addition of FHSO or KLX. The slopes of OHKLX and BuoKLX with added FHSO increased much slower than those with added KLX. This suggests that to increase hardness and at the same time keep the low slope value (high cohesiveness), FHSO can be effectively used. Similarly, for VTP ratio, adding FHSO to OHKLX and BuoKLX led to a slower decrease of VTP ratio than adding KLX to the two newly synthesized materials. Therefore, using FHSO in combination with OHKLX or BuoKLX will produce a wax material with proper hardness and cohesiveness. Both of these two properties should be considered when formulating wax materials for candles. From this study, the proper formulation should be about 60% OHKLX with 40% of FHSO for a hard wax. A mixture with about 70-90% of OHKLX and 10-30% of KLX will have hardness similar to Csoywax, and its cohesiveness should be significantly better than that of Csoywax.

Melting and crystallization properties. The melting and crystallization profiles of KLX and its derivatives are shown in FIG. 7. From the profiles, it seems the EPKLX did not have a significantly altered thermal properties compared to KLX, however, the BuoKLX had much lower melting and crystallization temperatures.

The temperatures of onset, peak, and completion of both the melting and crystallization peaks were measured. The temperature range of these peaks and the endothermic and exothermic energy (ΔH) were also calculated and these results are presented in Table 2 and FIGS. 8A-8B. Cparaffin and Beeswax absorbed and released more energy than others. The absorbed heat of Cparaffin, Beeswax, EPKLX, and FHSO during melting was similar to their released heat during crystallization. However, KLX, Csoywax, OHKLX, and BuoKLX needed more energy for melting than the energy released in crystallization. This may indicate that these materials do not crystallize as well and as fast as EPKLX and FHSO do, and crystallization may have continued during the low temperature treatment and storage. BuoKLX had the least energy exchange in a melting and recrystallization cycle, suggesting the most disordered crystalline packing. These energy data may provide insight about how easy the wax material will be melted to supply fuel for burning of candles and how readily the wax will solidify so it will not cause excessive dripping as seen in some vegetable oil candles.

As shown in Table 2, Csoywax had the lowest melting peak temperature while FHSO had the highest compared with KLX, EPKLX, and OHKLX. The melting peak temperature of BuoKLX was significantly lower than others, which agreed to the study reported by Sharma et al. (2006). They found that butyric ester of epoxidized soybean oil could be used as lubricant with a very low pour point.

The temperature range during melting and crystallization also were significantly different among paraffin wax, beeswax, Csoywax, and synthesized waxes (FIG. 8A). Beeswax had the widest melting range (62° C. for melting and 58° C. for crystallization) while FHSO had the narrowest range (17° C. for melting and 14° C. for recrystallization). The temperature ranges of Csoywax, KLX, EPKLX, and OHKLX were similar during melting, and they seem to be slightly wider than the range of Cparaffin. Proper melting and crystallization range is desired for liquid fuel formation and its containment. Candles made with materials with narrow melting range and high onset melting temperature tended to form holes or leaky channels which have been observed in FHSO. Meanwhile, candle with low onset melting temperature, such as very soft soywax, had dripping problems. Typically, material with a wide melting range is desirable for candle-making. The melting and crystallization profiles of selected formulation mixtures are shown in FIG. 9A-9B. The mixing of FHSO and KLX with OHKLX and BuoKLX led to increases in the temperature

TABLE 2
Melting and Crystallization of Bulk Waxes
(The first line of individual wax is the data for the main peak)

| | Melting | | | | | Crystallization | | | | |
|---|---|---|---|---|---|---|---|---|---|---|
| | Δ H, | Peak temp, | Onset | Complete | Temp | Δ H, | Peak | Onset | Complete | Temp |
| Wax | mJ/mg | ° C. | temp, ° C. | temp, ° C. | range, ° C. | mJ/mg | temp, ° C. | temp, ° C. | temp, ° C. | range, ° C. |
| Beeswax | 195.5 ± 0.5 | 65.1 ± 0.3 | 11.5 ± 1.6 | 73.7 ± 0.0 | 62.2 ± 1.6 | 197.8 ± 0.0 | 59.9 ± 0.1 | 63.0 ± 0.6 | 4.8 ± 0.1 | 58.3 ± 0.6 |
| Cparaffin | 206.6 ± 1.9 | 62.7 ± 0.4 | 30.2 ± 0.4 | 68.9 ± 1.0 | 38.8 ± 0.6 | 212.6 ± 4.2 | 56.4 ± 0.4 | 61.1 ± 0.2 | 27.3 ± 0.4 | 33.8 ± 0.1 |
| Csoywax | 110.6 ± 3.0 | 46.6 ± 0.6 | 14.9 ± 1.2 | 55.0 ± 1.3 | 40.2 ± 2.5 | 80.9 ± 2.8 | 32.6 ± 0.2 | 42.8 ± 0.2 | −2.3 ± 0.4 | 45.1 ± 0.6 |
| FHSO | 78.9 ± 13.9 | 55.8 ± 0.5 | 42.5 ± 0.9 | 59.6 ± 0.4 | 17.1 ± 1.3 | 140.2 ± 6.6 | 48.2 ± 0.8 | 51.0 ± 1.1 | 37.1 ± 0.1 | 14.0 ± 1.2 |
| | 77.6 ± 11.7 | 65.2 ± 0.4 | 59.6 ± 0.4 | 71.9 ± 1.1 | 12.4 ± 0.8 | | | | | |
| KLX | 119.1 ± 0.4 | 48.6 ± 0.4 | 20.3 ± 1.6 | 58.4 ± 0.0 | 38.1 ± 1.6 | 100.3 ± 2.7 | 35.7 ± 0.3 | 38.0 ± 0.0 | 2.1 ± 1.8 | 36.0 ± 1.8 |
| EPKLX | 99.1 ± 2.4 | 51.6 ± 1.3 | 8.2 ± 0.6 | 58.4 ± 1.8 | 50.2 ± 2.4 | 94.6 ± 3.2 | 36.3 ± 0.8 | 40.1 ± 1.5 | 13.1 ± 0.4 | 27.1 ± 1.1 |
| OHKLX | 79.7 ± 2.4 | 46.7 ± 1.3 | 7.4 ± 0.6 | 56.2 ± 1.8 | 48.8 ± 2.4 | 73.6 ± 3.2 | 35.8 ± 0.8 | 40.0 ± 1.5 | −1.9 ± 0.4 | 41.8 ± 1.1 |
| | 6.9 ± 0.3 | 75.7 ± 0.8 | 57.9 ± 0.6 | 79.8 ± 0.4 | 21.9 ± 0.1 | 4.9 ± 0.1 | 68.4 ± 1.9 | 72.3 ± 1.3 | 59.7 ± 5.0 | 4.9 ± 6.3 |
| BuoKLX | 38.9 ± 0.8 | 10.8 ± 2.3 | −26.2 ± 0.6 | 19.3 ± 0.6 | 45.5 ± 1.1 | 33.6 ± 0.6 | 7.4 ± 0.8 | 14.2 ± 0.2 | −27.4 ± 1.1 | 41.5 ± 1.3 |
| | 23.4 ± 3.3 | 37.9 ± 0.8 | 19.3 ± 0.6 | 41.8 ± 0.6 | 22.5 ± 1.2 | 14.0 ± 2.4 | 30.8 ± 1.8 | 35.8 ± 1.7 | 14.2 ± 0.2 | 21.7 ± 1.9 |
| | 13.6 ± 2.6 | 49.7 ± 0.2 | 43.0 ± 1.1 | 56.6 ± 3.1 | 13.6 ± 4.2 | | | | | |

range for both melting and crystallization cycles as shown in FIG. 9. Peak tailings were also observed in wax mixtures and the synthesized waxes. Narine et al (1999) reported fat peak tailing in their study of thermal properties of fat and suggested that the tailing indicated a gradual crystallization that may be attributed to disorder in a system. The total heat released or absorbed during crystallization and melting decreased significantly with the addition of OHKLX or BuoKLX to FHSO and KLX (FIGS. 10A-10B). The heat tended to level off as the content of OHKLX was greater than 50% in mixture, however, it decreased continuously with the addition of BuoKLX.

### REFERENCES

American Oil Chemists' Society, 4^{TH} edition, D. edited by Firestone, AOCS Press, Champaign, Ill. (1994).
Fine, P., and G. Cass. Characterization of fine particle emissions from burning church candles, Environ. Sci. Technol. 33:2352-2362 (1999).
Rezaei, K., T. Wang, and L. A. Johnson. Combustion characteristics of candle made from hydrogenated soybean oil, J. Am. Oil Chem. Soc. 79:803-808 (2002a).
Rezaei, K., T. Wang, and L. A. Johnson. Hydrogenated vegetable oils as candle wax. J. Am. Oil Chem. Soc. 79: 1241-1247 (2002b).
Edwards, R. T. Crystal habit of paraffin wax, Industrial and Engineering Chemistry 49: 750-757 10 (1957).
Dorset, D. L. Development of lamellar structures in natural waxes—an electron diffraction investigation, J. Phys. D: AppL. Phys. 32:1276-1280 (1999).
Dorset, D. L. The crystal structure of waxes, Acta Cryst. B 51:1021-1028 (1995).
Marangoni, A. G., and S. S. Narine. Rheology fundamentals and structural theory of elasticity, In Fat crystal networks, ed by Marangoni. A. G., Marcel Dekker, New York, 2005, 115-142.
Narine, S. S., and A. G. Marangoni. Fractal natural of fat crystal networks, Physical Review 59: 1908-1920 (1999).
Park, S., F. Jin, and J. Lee. Synthesis and thermal properties of epoxidized vegetable oil. Macromol. Rapid Commun. 25:724-727 (2004).
Sharma, B. K., A. Adhvaryu, Z. Liu, and S. Z. Erhan. Chemical modification of vegetable oils for lubricant applications. J. Am. Oil Chem. Soc. 83:129-136 (2006).
Swern, D. Organic peroxy acids as oxidizing agents. Epoxidation. In Organic Peroxides, Vol. 2, Willey-Interscience, NY, 1971, 355-533.
SAS, SAS Procedure Guide, Release 9.1, SAS institure, Inc., Cary, N.C., 2006.
Formo, M. W. Miscellaneous oil and fat products. In Bailey's Industrial Oil and Fat Products, 4^{th} ed. Swern, D., 1982, Wiley, New York, pp 366-371.
Rangarajan, B. H., E. A. Grulke, and P. D. Culnan. Kenetic parameters of a two-phase model for in situ epoxidation of soybean oil, J. Am. Oil Chem. Soc. 72:1161-1169 (1995).
Wright, A. J.; Scanlon, M. G.; Hartel, B. W.; Marangoni, A. G. Rheological properties of milkfat and butter, J. Food Sci. 66:1056-1071 (2001).

Persons skilled in the art will readily appreciate that the processes described above may in some instances be combined or separated into several steps. Further, persons skilled in the art will also readily appreciate that the processes of this invention may be accomplished using a variety of equipment and techniques that are well known in the art, including conveyor belts, chambers, condensers, centrifuges, distillers, etc. The specific equipment and processes used are not crucial so long as the intended result is accomplished.

It should be appreciated that minor modifications of the composition and the ranges expressed herein may be made and still come within the scope and spirit of the present invention.

Having described the invention with reference to particular compositions, theories of effectiveness, and the like, it will be apparent to those of skill in the art that it is not intended that the invention be limited by such illustrative embodiments or mechanisms, and that modifications can be made without departing from the scope or spirit of the invention, as defined by the appended claims. It is intended that all such obvious modifications and variations be included within the scope of the present invention as defined in the appended claims. The claims are meant to cover the claimed components and steps in any sequence which is effective to meet the objectives there intended, unless the context specifically indicates to the contrary.

What is claimed is:

1. A modified partially hydrogenated vegetable oil (PHVO) comprising: PHVO, said PHVO having a triacylglycerol chain, said PHVO having at least one functional group in the triacylglycerol chain.

2. The modified PHVO of claim 1 having better kneadability than unmodified PHVO.

3. The modified PHVO of claim 1 whereby the functional group is a hydroxyl group.

4. The modified PHVO of claim 1 that is selected from the group consisting of OHKLX, EPKLX, and BuoKLX.

5. A composition useful in the manufacture of candles, said composition comprising at least one modified partially hydrogenated vegetable oil (PHVO) having a triacylglycerol chain, said PHVO having at least one functional group in the triacylglycerol chain; and at least one unmodified PHVO.

6. The composition of claim 5 whereby the modified PHVO is selected from the group consisting of OHKLX, EPKLX, BuoKLX, and mixtures thereof.

7. The composition of claim 5 whereby the unmodified PHVO is selected from the group consisting of FHSO, KLX, and mixtures thereof.

8. The composition of claim 5 having better kneadability than unmodified PHVO, and better hardness than modified PHVO.

9. The composition of claim 5 comprising about 70-90% by weight OHKLX and about 10-30% by weight KLX.

10. The composition of claim 5 comprising about 60% OHKLX and about 40% FHSO.

11. A modified partially hydrogenated vegetable oil (PHVO) comprising: PHVO, said PHVO having a triacylglycerol chain, said PHVO having at least one hydroxyl group in the triacylglycerol chain.

12. A method of manufacturing a modified partially hydrogenated vegetable oil (PHVO) comprising: introducing at least one functional group into a triacylglycerol chain in a PHVO.

13. The method of claim 12 whereby the new functional group is a hydroxyl group.

14. The method of claim 12 whereby the hydroxyl group is introduced into the triacylglycerol chain by epoxidizing a PHVO to form an oxirane ring; opening the oxirane ring through a ring-opening reaction to form a dihydroxy compound; and esterifying the di-hydroxy compound to form the modified PHVO.

15. The method of claim 14 whereby the epoxidizing step comprises: combining the PHVO with an oxidant in the presence of a catalyst.

16. The method of claim 15 whereby the oxidant is hydrogen peroxide.

17. The method of claim 15 whereby the catalyst is a weak organic acid.

18. The method of claim 15 whereby the epoxidizing step is performed in the presence of a solvent.

19. The method of claim 14 whereby the ring-opening step comprises: combining the compound comprising the oxirane ring with a strong acid in aqueous solution.

20. The method of claim 14 whereby the esterifying step comprises: reacting the dihydroxy compound with a fatty acid entity capable of incorporating fatty acid acyl groups onto the triacylglycerol chain.

21. The method of claim 20 whereby the fatty acid entity is a short-chain fatty acid anhydride.

22. A method of manufacturing a compound useful in the manufacture of candles comprising: combining at least one modified partially hydrogenated vegetable oil (PHVO) having a triacylglycerol chain, said PHVO having at least one functional group in the triacylglycerol chain, with at least one unmodified PHVO.
